(19) United States
(12) Patent Application Publication
(10) Pub. No.: US 2026/0074051 A1
(43) Pub. Date: Mar. 12, 2026

(54) MENTAL HEALTH THERAPEUTIC TREATMENT METHOD

(71) Applicant: Emmalyne Laura Watt, Gatineau (CA)

(72) Inventor: Emmalyne Laura Watt, Gatineau (CA)

(21) Appl. No.: 18/826,786

(22) Filed: Sep. 6, 2024

(51) Int. Cl. G16H 20/70; G16H 10/60; G16H 80/00

(52) U.S. Cl. CPC G16H 20/70; G16H 10/60; G16H 80/00

(57) ABSTRACT

A mental health therapeutic treatment method that employs a mental health issue entity wherein the mental health issue entity is created utilizing generative computer techniques and represent an issue or a mental health issue that an individual desires to treat. The method provides collection of data between a practitioner and an individual wherein the data is input and analyzed in a thematic manner to identify parameters such as but not limited to thought patterns and trends. The software program is executed on computing hardware and provides generation of a mental health issue entity wherein the mental health issue entity is provided in either a visual or audial format via suitable computing equipment such as but not limited to display monitors. The mental health issue entity is provided in an interactive manner wherein the practitioner and individual engage in a therapy session with an interactive mental health issue entity.

### FIELD OF THE INVENTION

The present invention relates generally to mental healthcare, more specifically but not by way of limitation, a treatment method between a practitioner and an individual wherein the individual's mental health issue has data gathered thereon so as to produce a visual and/or audial representation of the mental health issue for subsequent engagement by the individual with the practitioner.

### BACKGROUND

Tens of millions of North Americans regularly visit a mental health practitioner which has resulted in a strong growth and acceptance of mental health counseling. Therapy of various types can be traced back several centuries wherein people have helped each other deal with mental health issues utilizing both informal and formal methodologies. People have been helping each other for centuries through talk, wellness practices, and meditation. Scholars agree that therapeutic-seeming practices have been documented since ancient Greece. However, “psychotherapy,” the official term for mental health therapy, officially became a term in the early 1800's. Before psychotherapy became popular, mental health conditions were often treated as curses, possessions, or hysteria. Many women were still treated for female hysteria utilizing rudimentary and sometimes painful techniques. Before the 1800s, individuals who experienced mental health symptoms or deviated from the norm in any way were commonly considered witches.

A practitioner/researcher by the name of Sigmund Freud did groundbreaking work in the field of clinical psychology which started the movement to make the field more accepted in the mainstream healthcare practice. Freud created psychoanalysis, which involved a therapist speaking to a patient one-on-one and analyzing their thought patterns, actions, and emotions. The approach to therapy laid the foundation for understanding psychological disorders and providing psychological treatments. The work of Freud and others in the field led to the development of psychodynamic therapy. Psychodynamic therapy delves into the unconscious and conscious aspects of the human mind. This approach opened up new possibilities for treating mental distress and mental illness. Modern psychologists and mental healthcare practitioners have taken the aspects of past therapy practices that work and applied them to modern science. Studies show that approximately seventy-five percent of people who engage in mental health therapies benefit from the therapy. Some psychologists believe biological factors influence mental health, many studies show that symptoms of most mental health conditions can be lessened or treated through talk therapy. While modern therapies have proven effective, improvements can be made in dealing with the mental health issues of the individual.

Accordingly, there is a need for a mental health therapeutic method that is executed between a practitioner and an individual wherein the method provides creation of a visual or audial representation of a particular mental health issue in order to provide an opportunity for the individual to engage with the representation of the mental health issue.

### SUMMARY OF THE INVENTION

It is the object of the present invention to provide a mental health therapeutic method executed between a practitioner and an individual wherein the method of the present invention includes identification of at least one mental health issue.

Another object of the present invention is to provide a method for treating mental health utilizing a representation of a mental health issue of an individual and enabling engagement therewith wherein the method of the present invention includes recordation of at least one therapy session between a practitioner and an individual wherein the recordation can be audio and/or visual.

A further object of the present invention is to provide a mental health therapeutic method executed between a practitioner and an individual wherein the present invention includes generative analytics of the recorded data.

Yet a further object of the present invention is to provide a method for treating mental health utilizing a representation of a mental health issue of an individual and enabling engagement therewith wherein the method of the present invention includes a generation of an audial and/or visual representation of at least one mental health issue identified from the recorded data.

Still another object of the present invention is to provide a mental health therapeutic method executed between a practitioner and an individual wherein the present invention includes projection of a visual representation of the mental health issue and further provides engagement therewith between the practitioner and individual.

An additional object of the present invention is to provide a method for treating mental health utilizing a representation of a mental health issue of an individual and enabling engagement therewith wherein the present invention includes at least on participative therapy session between the practitioner, individual and mental health issue generated entity that is displayed graphically to the individual.

Yet a further object of the present invention is to provide a mental health therapeutic method executed between a practitioner and an individual wherein the present invention displays the mental health issue generated entity in the likeness of the individual.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a flowchart of the method of the present invention.

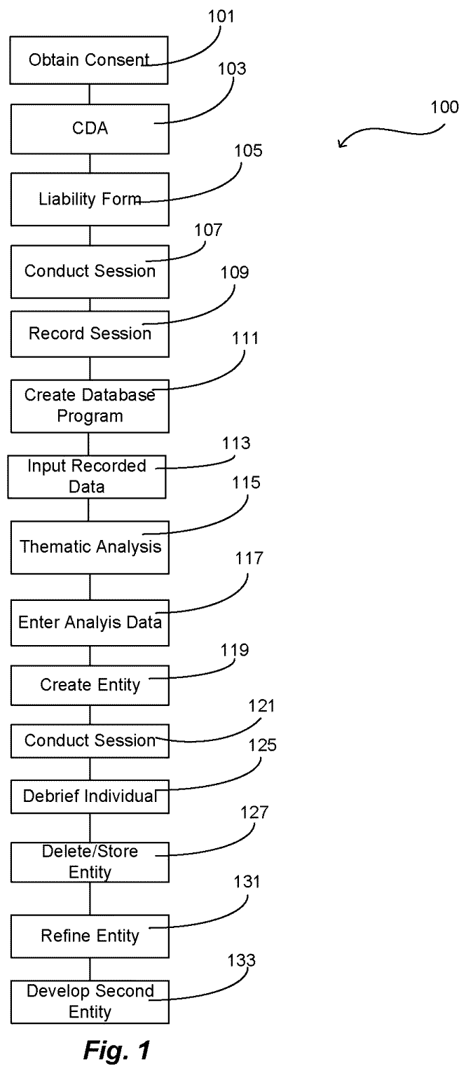

### DETAILED DESCRIPTION

Referring now to the drawings submitted herewith, wherein various elements depicted therein are not necessarily drawn to scale and wherein through the views and figures like elements are referenced with identical reference numerals, there is illustrated a mental health therapeutic treatment method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the mental health therapeutic treatment method 100 is diagrammed therein. It should be understood within the scope of the present invention that the steps outline herein are exemplary and the order thereof could be altered. It should be further understood that some of the steps illustrated and discussed herein could be eliminated and/or repeated. The mental health therapeutic treatment method 100 in its preferred embodiment is executed in a controlled environment between a healthcare practitioner and an individual.

The mental health therapeutic treatment method 100 initiates with step 101 wherein the healthcare practitioner obtains consent from an individual to participate in the mental health therapeutic treatment method 100. Step 103, the individual signs a confidentiality agreement. Step 105, a liability form is further completed between the practitioner and the individual. In step 107, the practitioner and the individual will engage in a mental health session. In practice, it is preferred that the mental health practitioner and the individual engage in multiple sessions. This would be important in order to develop the alliance, assess presenting issue, establish therapeutic goals, gather information through therapeutic dialogue and additional techniques. In step 109, the sessions conducted between the practitioner and the individual are recorded. It should be understood within the scope of the present invention that the recordings could be either video and/or audio recordings based on preference of the individuals. Both audial and visual recording are utilized to collect data such as but not limited to, mannerisms, emotional cues, intonation, descriptive features, physical features, images, photographs with the latter being employed for visual creation of the mental health issue entity representative further discussed herein.

In step 111, the operator of the mental health therapeutic treatment method 100 creates a software program utilizing conventional computing tools such as but not limited to desktop computers and databases. The software program of the present invention has data input thereinto pertaining to mental health conditions wherein the data is employed through generative techniques to provide generative analytics of the recorded data from the session(s) between the practitioner and individual. It should be understood within the scope of the present invention that generative analytics could employ artificial intelligence and/or be programmed execution of the software program of the present invention. The data contained in the software program of the present invention includes a database having data of psychological process, human development, psychology, mental illnesses, psychotherapy, all DSM versions, psychological interventions, pathological issues and mental health promotion strategies. In step 113 the practitioner will enter the data collected in the recorded session(s) between the practitioner and the individual. It is intrinsic to the present invention that the data collected in the session(s) between the practitioner and individual is stored on acceptable digital media in an accessible format.

Step 115, the software program of the present invention will perform a generative analytics of the recorded data collected from the session(s) between the practitioner and individual. The generative analytics comprises identifying patterns, trends and themes in order to provide insights, decision-making, predictive analytics and natural language processing most accurately depict the intended issue the individual desires to explore with the practitioner. This analysis enables the software program of the present invention to create new content in relation to the issue(s) being explored between the practitioner and the individual that could bring about new therapeutic insights, awareness and subconscious processing. In step 117, the generative analytics data is consolidated and processed by the software program of the present invention. This should be understood to include organization of identified mental health issues and similar activities.

In step 119, the software program of the present invention utilizes generative techniques to create a mental health issue entity wherein the mental health issue entity exhibits characteristics of the mental health issue identified in the generative analytics. The mental health issue entity is an interactive entity generated on a computing device wherein the computing device can display a visual representation of the mental health issue entity. It should be understood within the scope of the present invention that the graphical image of the mental health issue entity display can be the likeness of the individual or some other likeness. The graphical image of the mental health issue entity can be displayed utilizing visual image hardware such as but not limited to graphical display monitor, holographic generation device or image projection device. Furthermore, it should be understood within the scope of the present invention that the mental health issue entity could be presented in audio format with no graphical image provided. The software program provides the mental health issue entity and is configured to facilitate an interactive session between the mental health issue entity, individual and practitioner. Step 121, the practitioner and individual conduct at least one session wherein the session is conducted with an interactive mental health issue entity present in the session via computing techniques such as but not limited to audio and visual equipment. This step allows the individual to engage with the chosen therapeutic issue in a real-time as the therapeutic issue is represented by the mental health issue entity and the format provides an externalized manner to engage with the mental health issue/therapeutic issue of the individual. This allows for a greater level of psychological, cognitive, behavioral and emotional processing. The practitioner and individual dialogue with the mental health issue entity and come to greater insights and higher level of psychological processing.

In step 125, the practitioner debriefs with the individual subsequent completion of the session. This is important as it allows for the individual to share descriptions, thoughts, and feelings of the intervention thus facilitating healing and integration. Furthermore, the debrief can lead to greater insights and processing and may provide additional information that can be further incorporated into subsequent interactions with the mental health issue entity. Step 127, the practitioner can choose to store the mental health issue entity for further development and use in the software program of the present invention or delete the mental health issue entity if desired. In step 131, the software program can evaluate additional data collected from alternate sessions and provide refinement of the characteristics of the mental health issue entity for subsequent use thereof in future interactions. Step 133, if desired the practitioner and individual can employ the software program of the present invention to develop a second mental health issue entity wherein the second mental health issue entity represents an alternate mental health issue of the individual and can be utilized in sessions with the practitioner.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

1. A mental health therapeutic treatment method operable to provide an interactive engagement with a mental health issue of an individual during a therapy session wherein the method comprises:
obtaining consent, wherein a practitioner obtains consent from an individual to engage in therapy sessions of the mental health therapeutic treatment method;
providing at least one computer, wherein the at least one computer contains electronics to receive, store, transmit, display and manipulate data, said at least one computer having thereon an operational software program configured to be utilized in the mental health therapeutic treatment method;
conducting at least one session, wherein the practitioner conducts a therapy session with the individual;
recording the at least one session, wherein the at least one session between the practitioner and the individual is recorded creating a session data file;
inputting the session data file into the software program operating on the at least one computer;
executing generative analytics, wherein the session data file is reviewed by the software program for mental health parameters to establish generative analytics data;
entering the generative analytics data into the software program operating on the at least one computer;
creating an interactive mental health issue entity from the generative analytics data, wherein the software program creates the interactive mental health entity as a visual representation to be displayed or broadcast to the individual utilizing generative techniques;
conducting a subsequent therapy session, wherein the practitioner, the individual and the interactive mental health issue entity engage in a participative therapy session, wherein the interactive mental health issue entity is graphically displayed or audially broadcast to the individual on a graphical image production device;
debriefing the individual, wherein the practitioner debriefs the individual ensuing the subsequent therapy session.

2. The mental health therapeutic treatment method operable to provide an interactive engagement with a mental health issue of the individual as recited in claim 1, wherein the interactive mental health issue entity is a representation of a mental health issue on which the individual is focused in accordance with the generative analytics data.

3. The mental health therapeutic treatment method operable to provide an interactive engagement with a mental health issue of an individual as recited in claim 2, wherein a graphical display of the interactive mental health issue entity is in a likeness of the individual.

4. The mental health therapeutic treatment method operable to provide an interactive engagement with a mental health issue of an individual as recited in claim 3, and further including a step of collecting additional data, wherein the additional data is collected in extra therapy sessions.

5. The mental health therapeutic treatment method operable to provide an interactive engagement with a mental health issue of an individual as recited in claim 4, and further including a step of refining the interactive mental health issue entity, wherein the additional data is employed to provide refining of the interactive mental health issue entity.

6. The mental health therapeutic treatment method operable to provide an interactive engagement with a mental health issue of an individual as recited in claim 5, wherein the generative analytics comprises identifying patterns, trends and themes in order to provide insights, decision-making, predictive analytics and natural language processing.

7. The mental health therapeutic treatment method operable to provide an interactive engagement with a mental health issue of an individual as recited in claim 6, and further including a step of developing an additional interactive mental health issue entity, wherein the additional interactive mental health issue entity is based on a mental health parameter of the individual identified in the generative analytics which is different from the mental health issue of the interactive mental health issue entity.

8. The mental health therapeutic treatment method operable to provide an interactive engagement with a mental health issue of an individual as recited in claim 7, and further including a step of deleting or storing the interactive mental health issue entity to a computer-readable medium.
